(19) United States
(12) Patent Application Publication
(10) Pub. No.: US 2010/0113457 A1
(43) Pub. Date: May 6, 2010

(54) NOVEL PYRIDAZINE DERIVATIVES

(75) Inventors: Stephan Trah, Stein (CH); Clemens Lamberth, Stein (CH); Sebastian Wendeborn, Stein (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/447,025

(22) PCT Filed: Oct. 23, 2007
(86) PCT No.: PCT/EP2007/009188

§ 371 Date: Jan. 7, 2010

(30) Foreign Application Priority Data
Oct. 25, 2006 (EP) 06022292.4

(51) Int. Cl. A01N 43/58; C07D 237/12; C07D 237/14; A01P 3/00; A01P 1/00

(52) U.S. Cl. 514/247; 544/224; 544/241

(57) ABSTRACT

The present invention relates to novel pyridazine derivatives of formula (I) as active ingredients which have microbiocidal activity, in particular fungicidal activity: wherein R¹ is hydrogen, C₁-C₆alkyl, C₁-C₆haloalkyl or C₃-C₆cycloalkyl; R² is halogen, nitro, cyano, C₁-C₄alkyl, C₁-C₄haloalkyl, C₁-C₄alkoxy, C₁-C₄haloalkoxy, C₁-C₄alkylthio or C₁-C₄haloalkylthio; R³ is an optionally substituted aryl; R⁴ is fluoro, cyano, C₁-C₆haloalkyl, C₃-C₆cycloalkyl, C₁-C₆haloalkoxy, C₁-C₆alkylthio or C₁-C₆haloalkylthio; and n is a whole number from 1 to 4; or an agrochemically usable salt form thereof.

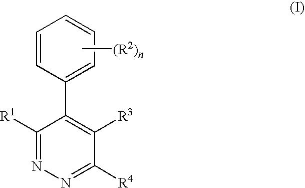

The present invention relates to novel pyridazine derivatives as active ingredients which have microbiocidal activity, in particular fungicidal activity. The invention also relates to preparation of these active ingredients to novel heterocyclic derivatives used as intermediates in the preparation of these active ingredients, to preparation of these novel intermediates, to agrochemical compositions which comprise at least one of the novel active ingredients, to preparation of these compositions and to use of the active ingredients or compositions in agriculture or horticulture for controlling or preventing infestation of plants, harvested food crops, seeds or non-living materials by phytopathogenic microorganisms, preferably fungi.

The present invention provides a compound of formula I:

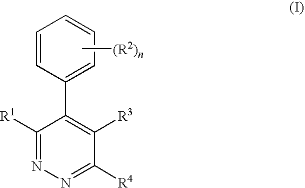

wherein
R¹ is hydrogen, C₁-C₆alkyl, C₁-C₆haloalkyl or C₃-C₆cycloalkyl;
R² is halogen, nitro, cyano, C₁-C₄alkyl, C₁-C₄haloalkyl, C₁-C₄alkoxy, C₁-C₄haloalkoxy, C₁-C₄alkylthio or C₁-C₄haloalkylthio;
R³ is an optionally substituted aryl;
R⁴ is fluoro, cyano, C₁-C₆haloalkyl, C₃-C₆cycloalkyl, C₁-C₆haloalkoxy, C₁-C₆alkylthio or C₁-C₆haloalkylthio; and
n is a whole number from 1 to 4;
or an agrochemically usable salt form thereof.

When n is 2, 3 or 4, then each R² independently of each other and independently at each occurrence can be the same or different.

In the above definition aryl includes aromatic hydrocarbon rings like phenyl, naphthyl, anthracenyl, phenanthrenyl and biphenyl, with phenyl being preferred.

The above aryl group and heteroaryl group may be optionally substituted. This means that they may carry one or more identical or different substituents. Normally not more than three substituents are present at the same time. Examples of substituents of aryl or heteroaryl groups are: halogen, alkyl, haloalkyl, cycloalkyl, cycloalkylalkyl, alkenyl, haloalkenyl, cycloalkenyl, alkynyl, haloalkynyl, alkyloxy, haloalkyloxy, cycloalkoxy, alkenyloxy, haloalkenyloxy, alkynyloxy, haloalkenyloxy, alkylthio, haloalkylthio, cycloalkylthio, alkenylthio, alkynylthio, alkylcarbonyl, haloalkylcarbonyl, cycloalkylcarbonyl, alkenylcarbonyl, alkynylcarbonyl, alkoxyalkyl, cyano, nitro, hydroxy, mercapto, amino, alkylamino, dialkylamino. Typical examples for optionally substituted aryl include 2-fluorophenyl, 2-chlorophenyl, 2-trifluoromethylphenyl, 2-methylphenyl, 2,3-difluorophenyl, 2,4-difluorophenyl, 2,5-difluorophenyl, 2,6-difluorophenyl, 2,3-dichlorophenyl, 2,4-dichlorophenyl, 2,5-dichlorophenyl, 2,6-dichlorophenyl, 2-chloro-3-fluorophenyl, 2-chloro-4-fluorophenyl, 2-chloro-5-fluorophenyl, 2-chloro-6-fluorophenyl, 3-chloro-2-fluorophenyl, 4-chloro-2-fluorophenyl, 5-chloro-2-fluorophenyl, 2-fluoro-3-trifluoromethylphenyl, 2-fluoro-4-trifluoromethylphenyl, 2-fluoro-5-trifluoromethylphenyl, 2-fluoro-6-trifluoromethylphenyl, 2-chloro-3-trifluoromethylphenyl, 2-chloro-4-trifluoromethylphenyl, 2-chloro-5-trifluoromethylphenyl, 2-chloro-6-trifluoromethylphenyl, 4-fluoro-2-trifluoromethylphenyl, 4-chloro-2-trifluoromethylphenyl, 2-fluoro-3-methylphenyl, 2-fluoro-4-methylphenyl, 2-fluoro-5-methylphenyl, 2-fluoro-6-methylphenyl, 2-chloro-3-methylphenyl, 2-chloro-4-methylphenyl, 2-chloro-5-methylphenyl, 2-chloro-6-methylphenyl, 4-fluoro-2-methylphenyl, 4-chloro-2-methylphenyl, 2,4,6-trifluorophenyl, 2,3,6-trifluorophenyl, 2,3,4-trifluorophenyl, 2,4,6-trichlorophenyl, 2,3,6-trichlorophenyl, 2,3,4-trichlorophenyl, 2,6-difluoro-4-methoxyphenyl, 2,6-difluoro-4-trifluoromethoxyphenyl, 2,6-difluoro-4-trifluoromethylphenyl, 2,6-difluoro-4-cyanophenyl, 2,6-difluoro-4-methylphenyl, 2,6-dichloro-4-methoxyphenyl, 2,6-dichloro-4-trifluoromethoxyphenyl, 2,6-dichloro-4-trifluoromethylphenyl, 2,6-dichloro-4-cyanophenyl, 2,6-dichloro-4-methylphenyl, pentafluorophenyl.

In the above definition halogen is fluorine, chlorine, bromine or iodine.

The alkyl, alkenyl or alkynyl radicals may be straight-chained or branched.

Alkyl on its own or as part of another substituent is, depending upon the number of carbon atoms mentioned, for example, methyl, ethyl, propyl, butyl, pentyl, hexyl and the isomers thereof, for example, isopropyl, isobutyl, sec-butyl, tert-butyl, isopentyl or tert-pentyl.

A haloalkyl group may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Cycloalkyl on its own or as part of another substituent is, depending upon the number of carbon atoms mentioned, for example, cyclopropyl, cyclobutyl, cyclopentyl or cyclohexyl.

Alkenyl on its own or as part of another substituent is, depending upon the number of carbon atoms mentioned, for example, ethenyl, allyl, 1-propenyl, buten-2-yl, buten-3-yl, penten-1-yl, penten-3-yl, hexen-1-yl or 4-methyl-3-pentenyl.

Alkynyl on its own or as part of another substituent is, depending upon the number of carbon atoms mentioned, for example, ethynyl, propyn-1-yl, propyn-2-yl, butyn-1-yl, butyn-2-yl, 1-methyl-2-butynyl, hexyn-1-yl or 1-ethyl-2-butynyl.

The presence of one or more possible asymmetric carbon atoms in a compound of formula I means that the compounds may occur in optically isomeric, that means enantiomeric or diastereomeric forms. As a result of the presence of a possible aliphatic C═C double bond, geometric isomerism, that means cis-trans or (E)-(Z) isomerism may also occur. Also atropisomers may occur as a result of restricted rotation about a single bond. Formula I is intended to include all those possible isomeric forms and mixtures thereof. The present invention intends to include all those possible isomeric forms and mixtures thereof for a compound of formula I.

In each case, the compounds of formula I according to the invention are in free form or in an agronomically usable salt form.

In a first embodiment, compounds of formula I according to the invention have R¹ which is C₁-C₆alkyl, C₁-C₆haloalkyl or C₃-C₆cycloalkyl.

In a second embodiment, compounds of formula I according to the invention have R² which is halogen, nitro, cyano, C₁-C₃alkyl, C₁-C₃haloalkyl, C₁-C₃alkoxy, C₁-C₃haloalkoxy, C₁-C₃alkylthio or C₁C₃haloalkylthio.

In a third embodiment, compounds of formula I according to the invention have R³ which is an optionally substituted phenyl

In a fourth embodiment, compounds of formula I according to the invention have R⁴ which is fluoro, cyano, C₁-C₆haloalkyl, C₁-C₆haloalkoxy, C₁-C₆alkylthio or C₁-C₆haloalkylthio.

In a fifth embodiment, compounds of formula I according to the invention have n which is a whole number from 1 to 3.

Preferred subgroups of compounds of formula I according to the invention are those wherein

R¹ is C₁-C₆alkyl or C₁-C₆haloalkyl;
R² is halogen, nitro, cyano, C₁-C₂alkyl, C₁-C₂haloalkyl, C₁-C₂alkoxy, C₁-C₂haloalkoxy, C₁-C₂alkylthio or C₁-C₂haloalkylthio;
R³ is 2-fluorophenyl, 2-chlorophenyl, 2-trifluoromethylphenyl, 2-methylphenyl, 2,3-difluorophenyl, 2,4-difluorophenyl, 2,5-difluorophenyl, 2,6-difluorophenyl; 2,3-dichlorophenyl, 2,4-dichlorophenyl, 2,5-dichlorophenyl, 2,6-dichlorophenyl, 2-chloro-3-fluorophenyl; 2-chloro-4-fluorophenyl, 2-chloro-5-fluorophenyl, 2-chloro-6-fluorophenyl, 3-chloro-2-fluorophenyl, 4-chloro-2-fluorophenyl, 5-chloro-2-fluorophenyl, 2-fluoro-3-trifluoromethylphenyl, 2-fluoro-4-trifluoromethylphenyl, 2-fluoro-5-trifluoromethylphenyl, 2-fluoro-6-trifluoromethylphenyl, 2-chloro-3-trifluoromethylphenyl, 2-chloro-4-trifluoromethylphenyl, 2-chloro-5-trifluoromethylphenyl, 2-chloro-6-trifluoromethylphenyl, 4-fluoro-2-trifluoromethylphenyl, 4-chloro-2-trifluoromethylphenyl, 2-fluoro-3-methylphenyl, 2-fluoro-4-methylphenyl, 2-fluoro-5-methylphenyl, 2-fluoro-6-methylphenyl, 2-chloro-3-methylphenyl, 2-chloro-4-methylphenyl, 2-chloro-5-methylphenyl, 2-chloro-6-methylphenyl, 4-fluoro-2-methylphenyl, 4-chloro-2-methylphenyl, 2,4,6-trifluorophenyl, 2,3,6-trifluorophenyl, 2,3,4-trifluorophenyl, 2,4,6-trichlorophenyl, 2,3,6-trichlorophenyl, 2,3,4-trichlorophenyl, 2,6-difluoro-4-methoxyphenyl, 2,6-difluoro-4-trifluoromethoxyphenyl, 2,6-difluoro-4-trifluoromethylphenyl, 2,6-difluoro-4-cyanophenyl, 2,6-difluoro-4-methylphenyl, 2,6-dichloro-4-methoxyphenyl, 2,6-dichloro-4-trifluoromethoxyphenyl, 2,6-dichloro-4-trifluoromethylphenyl, 2,6-dichloro-4-cyanophenyl, 2,6-dichloro-4-methylphenyl or pentafluorophenyl;
R⁴ is fluoro, cyano or C₁-C₆haloalkoxy; and
n is a whole number from 1 to 2.

More preferred subgroups of compounds of formula I according to the invention are those wherein

R¹ is C₁-C₃alkyl;
R² is chloro, bromo, fluoro, nitro, cyano, methyl, C₁-C₂haloalkyl, methoxy, trifluoromethoxy, difluoromethoxy, chloro-difluoromethoxy, bromo-difluoromethoxy, methylthio or trifluoromethylthio;
R³ is 2-trifluoromethylphenyl, 2,4-dichlorophenyl, 2-chloro-6-fluorophenyl, 2,4,6-trifluorophenyl or 2,6-difluoro-4-methoxyphenyl;
R⁴ is fluoro, cyano or 2,2,2-trifluoroethoxy; and
n is a whole number from 1 to 2.

Most preferred subgroups of compounds of formula I according to the invention are those wherein

R¹ is methyl;
R² chloro;
R³ is 2,4,6-trichlorophenyl;
R⁴ is fluoro or 2,2,2-trifluoroethoxy; and
n is 1.

Preferred individual compounds are:
4-(4-chlorophenyl)-6-fluoro-3-methyl-5-(2,4,6-trifluorophenyl)-pyridazine, 3-fluoro-6-methyl-5-p-tolyl-4-(2,4,6-trifluorophenyl)-pyridazine,
5-(4-chlorophenyl)-6-methyl-4-(2,4,6-trifluorophenyl)-pyridazine-3-carbonitrile,
6-methyl-5-p-tolyl-4-(2,4,6-trifluorophenyl)-pyridazine-3-carbonitrile,
4-(4-chlorophenyl)-3-methyl-6-(2,2,2-trifluoroethoxy)-5-(2,4,6-trifluorophenyl)-pyridazine,
3-methyl-4-p-tolyl-6-(2,2,2-trifluoroethoxy)-5-(2,4,6-trifluorophenyl)-pyridazine,
4-(2-chloro-6-fluorophenyl)-5-(4-chlorophenyl)-3-fluoro-6-methyl-pyridazine,
4-(2-chloro-6-fluorophenyl)-3-fluoro-6-methyl-5-p-tolyl-pyridazine,
4-(2-chloro-6-fluorophenyl)-5-(4-chlorophenyl)-6-methyl-pyridazine-3-carbonitrile,
4-(2-chloro-6-fluorophenyl)-6-methyl-5-p-tolyl-pyridazine-3-carbonitrile,
4-(2-chloro-6-fluorophenyl)-5-(4-chlorophenyl)-6-methyl-3-(2,2,2-trifluoroethoxy)-pyridazine, and
4-(2-chloro-6-fluorophenyl)-6-methyl-5-p-tolyl-3-(2,2,2-trifluoroethoxy)-pyridazine.

Certain pyridazine derivatives with chloro-, bromo-, alkyl- or alkoxy-substituents in positions 3 and/or 6 have been proposed for controlling plant-destructive fungi, for example in WO 2005/121104 and WO 2006/001175. However, the action of those preparations is not satisfactory in all aspects of agricultural needs. Surprisingly, with the compounds of formula I, new kinds of fungicides having a high level of biological activity have now been found.

The compounds of formula I.2, wherein R¹, R², R³ and n are as defined for compound of formula I, can be obtained by transformation of a compound of formula I.1, wherein R¹, R², R³ and n are as defined for compound of formula I and Hal is chlorine or bromine, with a inorganic fluoride, e.g. potassium fluoride.

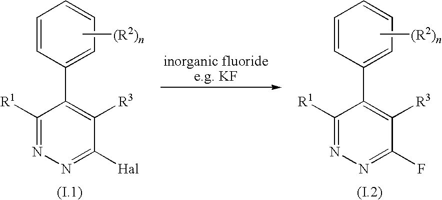

The compounds of formula I.3, wherein R¹, R², R³ and n are as defined for compound of formula I, can be obtained by transformation of a compound of formula I.1, wherein R¹, R², R³ and n are as defined for compound of formula I and Hal is chlorine or bromine, with a inorganic cyanide, e.g. sodium cyanide, potassium cyanide or copper cyanide.

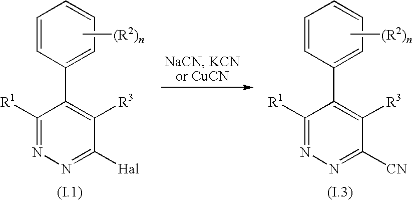

The compounds of formula I.4, wherein R¹, R², R³ and n are as defined for compound of formula I and R⁵ is C₁-C₆haloalkyl, can be obtained by reaction of a compound of formula I.1, wherein R¹, R², R³ and n are as defined for compound of formula I and Hal is halogen, preferably fluorine, chlorine or bromine, with an alcohol R⁵OH, wherein R⁵ is C₁-C₆haloalkyl, and a base or with a sodium alkoxide NaOR⁵, wherein R⁵ is C₁-C₆haloalkyl.

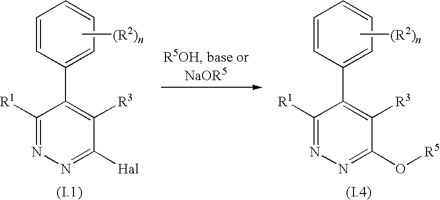

The compounds of formula I.5, wherein R¹, R², R³ and n are as defined for compound of formula I and R⁵ is C₁-C₆alkyl or C₁-C₆haloalkyl, can be obtained by reaction of a compound of formula I.1, wherein R¹, R², R³ and n are as defined for compound of formula I and Hal is halogen, preferably fluorine, chlorine or bromine, with a thiol R⁵SH, wherein R⁵ is C₁-C₆alkyl or C₁-C₆haloalkyl, and a base or with a sodium thioalkoxide NaSR⁵, wherein R⁵ is C₁-C₆alkyl or C₁-C₆haloalkyl.

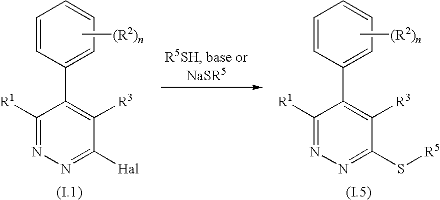

Surprisingly, it has now been found that the novel compounds of formula I have, for practical purposes, a very advantageous spectrum of activities for protecting plants against diseases that are caused by fungi as well as by bacteria and viruses.

The compounds of formula I can be used in the agricultural sector and related fields of use as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and are used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula I before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds according to present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula I are, for example, effective against the phytopathogenic fungi of the following classes: Fungi imperfecti (e.g. Botrytis spp., Alternaria spp.) and Basidiomycetes (e.g. Rhizoctonia spp., Hemileia spp., Puccinia spp., Phakopsora spp., Ustilago spp., Tilletia spp.). Additionally, they are also effective against Ascomycetes (e.g. Venturia spp., Blumeria spp., Podosphaera leucotricha, Monilinia spp., Fusarium spp., Uncinula spp., Mycosphaerella spp., Pyrenophora spp., Rhynchosporium secalis, Magnaporthe spp., Colletotrichum spp., Gaeumannomyces graminis, Tapesia spp., Ramularia spp., Microdochium nivale, Sclerotinia spp.) and Oomycetes (e.g. Phytophthora spp., Pythium spp., Plasmopara spp., Pseudoperonospora cubensis). Outstanding activity has been observed against powdery mildews (e.g. Uncinula necator), rusts (e.g. Puccinia spp.) and leaf spots (e.g. Septoria tritici). Furthermore, the novel compounds of formula I are effective against phytopathogenic bacteria and viruses (e.g. against Xanthomonas spp, Pseudomonas spp, Erwinia amylovora as well as against the tobacco mosaic virus).

Within the scope of present invention, target crops to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as turf and ornamentals.

The target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The compounds of formula I are used in unmodified form or, preferably, together with the adjuvants conventionally employed in the art of formulation. To this end they are conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula I are normally used in the form of fungicidal compositions for controlling or protecting against phytopathogenic microorganisms, comprising as active ingredient at least one compound of formula I, in free form or in agrochemically usable salt form, and at least one of the above-mentioned adjuvants.

The compounds of formula I can be mixed with other fungicides, resulting in some cases in unexpected synergistic activities. Mixing components which are particularly preferred are:

Azoles, such as azaconazole, BAY 14120, bitertanol, bromuconazole, cyproconazole, difenoconazole, diniconazole, epoxiconazole, fenbuconazole, fluquinconazole, flusilazole, flutriafol, hexaconazole, imazalil, imibenconazole, ipconazole, metconazole, myclobutanil, pefurazoate, penconazole, prothioconazole, pyrifenox, prochloraz, propiconazole, simeconazole, tebuconazole, tetraconazole, triadimefon, triadimenol, triflumizole, triticonazole;

Pyrimidinyl carbinoles, such as ancymidol, fenarimol, nuarimol;

2-amino-pyrimidines, such as bupirimate, dimethirimol, ethirimol;

Morpholines, such as dodemorph, fenpropidine, fenpropimorph, spiroxamine, tridemorph;

Anilinopyrimidines, such as cyprodinil, mepanipyrim, pyrimethanil;

Pyrroles, such as fenpiclonil, fludioxonil;

Phenylamides, such as benalaxyl, furalaxyl, metalaxyl, R-metalaxyl, ofurace, oxadixyl;

Benzimidazoles, such as benomyl, carbendazim, debacarb, fuberidazole, thiabendazole;

Dicarboximides, such as chlozolinate, dichlozoline, iprodione, myclozoline, procymidone, vinclozoline;

Carboxamides, such as boscalid, carboxin, fenfuram, flutolanil, mepronil, oxycarboxin, penthiopyrad, thifluzamide; guanidines, such as guazatine, dodine, iminoctadine;

Strobilurines, such as azoxystrobin, dimoxystrobin, enestroburin, fluoxastrobin, kresoxim-methyl, metominostrobin, trifloxystrobin, orysastrobin, picoxystrobin, pyraclostrobin;

Dithiocarbamates, such as ferbam, mancozeb, maneb, metiram, propineb, thiram, zineb, ziram;

N-halomethylthiotetrahydrophthalimides, such as captafol, captan, dichlofluanid, fluoromides, folpet, tolyfluanid;

Cu-compounds, such as Bordeaux mixture, copper hydroxide, copper oxychloride, copper sulfate, cuprous oxide, mancopper, oxine-copper;

Nitrophenol-derivatives, such as dinocap, nitrothal-isopropyl;

Organo-phosphorus-derivatives, such as edifenphos, iprobenphos, isoprothiolane, phosdiphen, pyrazophos, tolclofos-methyl;

Pyridazine-derivatives which are known and may be prepared by methods as described in WO 05/121104, WO 06/001175 and WO 07/066,601, such as 3-chloro-5-(4-chloro-phenyl)-6-methyl-4-(2,4,6-trifluoro-phenyl)-pyridazine (formula P.1), 3-chloro-6-methyl-5-p-tolyl-4-(2,4,6-trifluoro-phenyl)-pyridazine (formula P.2) and 3-chloro-4-(3-chloro-5-methoxy-pyridin-2-yl)-5-(4-chloro-phenyl)-6-methyl-pyridazine (formula P.3);

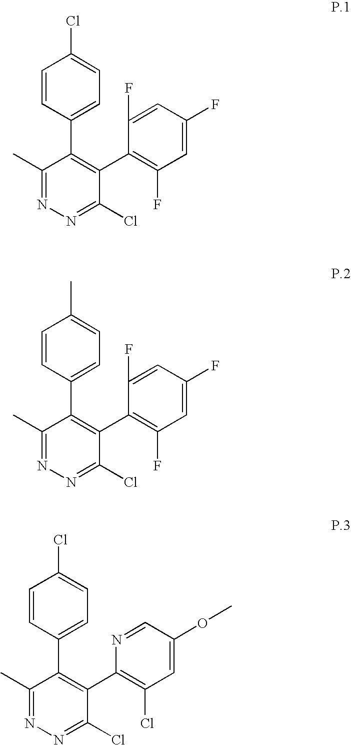

Triazolopyrimidine derivatives which are known and may be prepared by methods as described in WO98/46607, such as 5-chloro-7-(4-methyl-piperidin-1-yl)-6-(2,4,6-trifluorophenyl)-[1,2,4]triazolo[1,5-a]pyrimidine (formula T.1);

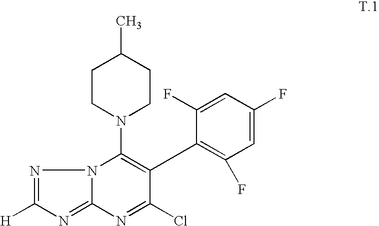

Carboxamide derivatives which are known and may be prepared by methods as described in WO04/035589 and in WO06/37632, such as 3-difluoromethyl-1-methyl-1H-pyrazole-4-carboxylic acid (9-isopropyp-1,2,3,4-tetrahaydro-1,4-methano-naphthalen-5-yl)-amide (formula U.1); or

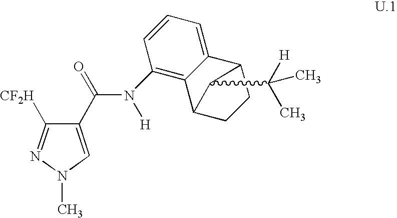

N-(3′,4′-dichloro-5-fluoro-1,1′-biphenyl-2-yl)-3-(difluoromethyl)-1-methyl-1H-pyrazole-4-carboxamide (compound F-13)

Benzamide derivatives which are known and may be prepared by methods as described in WO 2004/016088, such as N-{-2-[3-chloro-5-(trifluoromethyl)-2-pyridinyl]ethyl}-2-trifluoromethylbenzamide, which is also known under the name fluopyram (formula V.1);

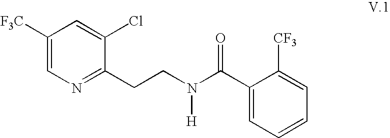

and

Various others, such as acibenzolar-5-methyl, anilazine, benthiavalicarb, blasticidin-S, chinomethionate, chloroneb, chlorothalonil, cyflufenamid, cymoxanil, dichlone, diclocymet, diclomezine, dicloran, diethofencarb, dimethomorph, flumorph, dithianon, ethaboxam, etridiazole, famoxadone, fenamidone, fenoxanil, fentin, ferimzone, fluazinam, fluopicolide, flusulfamide, fenhexamid, fosetyl-aluminium, hymexazol, iprovalicarb, cyazofamid, kasugamycin, mandipropamid, methasulfocarb, metrafenone, nicobifen, pencycuron, phthalide, polyoxins, probenazole, propamocarb, proquinazid, pyroquilon, quinoxyfen, quintozene, sulfur, tiadinil, triazoxide, tricyclazole, triforine, validamycin, zoxamide and glyphosate.

Another aspect of invention is related to the use of a compound of formula I, of a composition comprising at least one compound of formula I or of a fungicidal mixture comprising at least one compound of formula I in admixture with other fungicides, as described above, for controlling or preventing infestation of plants, harvested food crops or non-living materials by phytopathogenic microorganisms, preferably fungal organisms.

A further aspect of invention is related to a method of controlling or preventing an infestation of crop plants or of non-living materials by phytopathogenic or spoilage microorganisms or organisms potentially harmful to man, especially fungal organisms, which comprises the application of a compound of formula I as active ingredient to the plants, to parts of the plants or to the locus thereof, or to any part of the non-living materials. Controlling or preventing means reducing the infestation of crop plants or of non-living materials by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula I, or an agrochemical composition which contains at least one of said compounds, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation [that is, a composition containing the compound of formula I] and, if desired, a solid or liquid adjuvant or monomers for encapsulating the compound of formula I, is prepared in a known manner, typically by intimately mixing and/or grinding the compound with extenders, for example solvents, solid carriers and, optionally, surface active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

Whereas it is preferred to formulate commercial products as concentrates, the end user will normally use dilute formulations.

The following non-limiting examples illustrate the above-described invention in more detail.

### EXAMPLE 1

### This example illustrates the preparation of 4-(4-chlorophenyl)-6-fluoro-3-methyl-5-(2,4,6-trifluorophenyl)-pyridazine (Compound No.I.c.127)

A mixture of 4-(4-chlorophenyl)-6-chloro-3-methyl-5-(2,4,6-trifluorophenyl)-pyridazine (known from WO 2005/121104, 0.25 g), potassium fluoride (0.1 g) and 10 ml of dimethyl sulfoxide (DMSO) is heated to 140° C. for 72 h. After cooling, brine is added and the reaction mixture is then extracted with tert-butyl methyl ether. The combined organic layer is washed with brine and water, dried over sodium sulfate and evaporated under reduced pressure. The residue is purified by chromatography on silica gel, using a mixture of hexane/ethyl acetate 4:1 as eluent, to deliver 4-(4-chlorophenyl)-6-fluoro-3-methyl-5-(2,4,6-trifluorophenyl)-pyridazine (Compound No.I.c.127) as colourless crystals (from hexane), m.p. 114-115° C.

### EXAMPLE 2

### This Example Illustrates the Preparation of 4-(4-chlorophenyl)-3-methyl-6-(2,2,2-trifluoroethoxy)-5-(2,4,6-trifluorophenyl)-pyridazine (Compound No.I.c.129)

A mixture of 4-(4-chlorphenyl)-6-chloro-3-methyl-5-(2,4,6-trifluorophenyl)-pyridazine (known from WO 2005/121104, 0.31 g), potassium carbonate (0.13 g) and 5 ml of 2,2,2-trifluoroethanol is heated to reflux for 24 h. Subsequently the reaction mixture is cooled, diluted with water and extracted with ethyl acetate. The combined organic layer is washed with water and brine, dried over sodium sulfate and evaporated under reduced pressure. The remainder is purified by chromatography on silica gel, using a mixture of hexane/dichloromethane 2:1 as eluent to obtain 4-(4-chlorophenyl)-3-methyl-6-(2,2,2-trifluoroethoxy)-5-(2,4,6-trifluorophenyl)-pyridazine (Compound No.I.c.129) as a solid, m.p. 105-108° C.

Table 1 below illustrates examples of individual compounds of formula I according to the invention.

TABLE 1
individual compounds of formula I according to the invention

| Compound | | | |
|---|---|---|---|
| No. | R¹ | R³ | R⁴ |
| 001 | CH₃ | 2-fluorophenyl | F |
| 002 | CH₃ | 2-fluorophenyl | CN |
| 003 | CH₃ | 2-fluorophenyl | OCH₂CF₃ |
| 004 | CH₃ | 2-chlorophenyl | F |
| 005 | CH₃ | 2-chlorophenyl | CN |
| 006 | CH₃ | 2-chlorophenyl | OCH₂CF₃ |
| 007 | CH₃ | 2-trifluoromethylphenyl | F |
| 008 | CH₃ | 2-trifluoromethylphenyl | CN |
| 009 | CH₃ | 2-trifluoromethylphenyl | OCH₂CF₃ |
| 010 | CH₃ | 2-methylphenyl | F |
| 011 | CH₃ | 2-methylphenyl | CN |
| 012 | CH₃ | 2-methylphenyl | OCH₂CF₃ |
| 013 | CH₃ | 2,3-difluorophenyl | F |
| 014 | CH₃ | 2,3-difluorophenyl | CN |
| 015 | CH₃ | 2,3-difluorophenyl | OCH₂CF₃ |
| 016 | CH₃ | 2,4-difluorophenyl | F |
| 017 | CH₃ | 2,4-difluorophenyl | CN |
| 018 | CH₃ | 2,4-difluorophenyl | OCH₂CF₃ |
| 019 | CH₃ | 2,5-difluorophenyl | F |
| 020 | CH₃ | 2,5-difluorophenyl | CN |
| 021 | CH₃ | 2,5-difluorophenyl | OCH₂CF₃ |
| 022 | CH₃ | 2,6-difluorophenyl | F |
| 023 | CH₃ | 2,6-difluorophenyl | CN |
| 024 | CH₃ | 2,6-difluorophenyl | OCH₂CF₃ |
| 025 | CH₃ | 2,3-dichlorophenyl | F |
| 026 | CH₃ | 2,3-dichlorophenyl | CN |
| 027 | CH₃ | 2,3-dichlorophenyl | OCH₂CF₃ |
| 028 | CH₃ | 2,4-dichlorophenyl | F |
| 029 | CH₃ | 2,4-dichlorophenyl | CN |
| 030 | CH₃ | 2,4-dichlorophenyl | OCH₂CF₃ |
| 031 | CH₃ | 2,5-dichlorophenyl | F |
| 032 | CH₃ | 2,5-dichlorophenyl | CN |
| 033 | CH₃ | 2,5-dichlorophenyl | OCH₂CF₃ |
| 034 | CH₃ | 2,6-dichlorophenyl | F |
| 035 | CH₃ | 2,6-dichlorophenyl | CN |
| 036 | CH₃ | 2,6-dichlorophenyl | OCH₂CF₃ |
| 037 | CH₃ | 2-chloro-3-fluorophenyl | F |
| 038 | CH₃ | 2-chloro-3-fluorophenyl | CN |
| 039 | CH₃ | 2-chloro-3-fluorophenyl | OCH₂CF₃ |
| 040 | CH₃ | 2-chloro-4-fluorophenyl | F |
| 041 | CH₃ | 2-chloro-4-fluorophenyl | CN |
| 042 | CH₃ | 2-chloro-4-fluorophenyl | OCH₂CF₃ |
| 043 | CH₃ | 2-chloro-5-fluorophenyl | F |
| 044 | CH₃ | 2-chloro-5-fluorophenyl | CN |
| 045 | CH₃ | 2-chloro-5-fluorophenyl | OCH₂CF₃ |
| 046 | CH₃ | 2-chloro-6-fluorophenyl | F |
| 047 | CH₃ | 2-chloro-6-fluorophenyl | CN |
| 048 | CH₃ | 2-chloro-6-fluorophenyl | OCH₂CF₃ |
| 049 | CH₃ | 3-chloro-2-fluorophenyl | F |
| 050 | CH₃ | 3-chloro-2-fluorophenyl | CN |
| 051 | CH₃ | 3-chloro-2-fluorophenyl | OCH₂CF₃ |
| 052 | CH₃ | 4-chloro-2-fluorophenyl | F |
| 053 | CH₃ | 4-chloro-2-fluorophenyl | CN |
| 054 | CH₃ | 4-chloro-2-fluorophenyl | OCH₂CF₃ |
| 055 | CH₃ | 5-chloro-2-fluorophenyl | F |
| 056 | CH₃ | 5-chloro-2-fluorophenyl | CN |
| 057 | CH₃ | 5-chloro-2-fluorophenyl | OCH₂CF₃ |
| 058 | CH₃ | 2-fluoro-3-trifluoromethylphenyl | F |
| 059 | CH₃ | 2-fluoro-3-trifluoromethylphenyl | CN |
| 060 | CH₃ | 2-fluoro-3-trifluoromethylphenyl | OCH₂CF₃ |
| 061 | CH₃ | 2-fluoro-4-trifluoromethylphenyl | F |
| 062 | CH₃ | 2-fluoro-4-trifluoromethylphenyl | CN |
| 063 | CH₃ | 2-fluoro-4-trifluoromethylphenyl | OCH₂CF₃ |
| 064 | CH₃ | 2-fluoro-5-trifluoromethylphenyl | F |
| 065 | CH₃ | 2-fluoro-5-trifluoromethylphenyl | CN |
| 066 | CH₃ | 2-fluoro-5-trifluoromethylphenyl | OCH₂CF₃ |
| 067 | CH₃ | 2-fluoro-6-trifluoromethylphenyl | F |
| 068 | CH₃ | 2-fluoro-6-trifluoromethylphenyl | CN |
| 069 | CH₃ | 2-fluoro-6-trifluoromethylphenyl | OCH₂CF₃ |
| 070 | CH₃ | 2-chloro-3-trifluoromethylphenyl | F |
| 071 | CH₃ | 2-chloro-3-trifluoromethyiphenyl | CN |
| 072 | CH₃ | 2-chloro-3-trifluoromethylphenyl | OCH₂CF₃ |
| 073 | CH₃ | 2-chloro-4-trifluoromethylphenyl | F |
| 074 | CH₃ | 2-chloro-4-trifluoromethylphenyl | CN |
| 075 | CH₃ | 2-chloro-4-trifluoromethylphenyl | OCH₂CF₃ |
| 076 | CH₃ | 2-chloro-5-trifluoromethylphenyl | F |
| 077 | CH₃ | 2-chloro-5-trifluoromethylphenyl | CN |
| 078 | CH₃ | 2-chloro-5-trifluoromethylphenyl | OCH₂CF₃ |
| 079 | CH₃ | 2-chloro-6-trifluoromethylphenyl | F |
| 080 | CH₃ | 2-chloro-6-trifluoromethylphenyl | CN |
| 081 | CH₃ | 2-chloro-6-trifluoromethylphenyl | OCH₂CF₃ |
| 082 | CH₃ | 4-fluoro-2-trifluoromethylphenyl | F |
| 083 | CH₃ | 4-fluoro-2-trifluoromethylphenyl | CN |
| 084 | CH₃ | 4-fluoro-2-trifluoromethylphenyl | OCH₂CF₃ |
| 085 | CH₃ | 4-chloro-2-trifluoromethylphenyl | F |
| 086 | CH₃ | 4-chloro-2-trifluoromethylphenyl | CN |
| 087 | CH₃ | 4-chloro-2-trifluoromethylphenyl | OCH₂CF₃ |
| 088 | CH₃ | 2-fluoro-3-methylphenyl | F |
| 089 | CH₃ | 2-fluoro-3-methylphenyl | CN |
| 090 | CH₃ | 2-fluoro-3-methylphenyl | OCH₂CF₃ |
| 091 | CH₃ | 2-fluoro-4-methylphenyl | F |
| 092 | CH₃ | 2-fluoro-4-methylphenyl | CN |
| 093 | CH₃ | 2-fluoro-4-methylphenyl | OCH₂CF₃ |
| 094 | CH₃ | 2-fluoro-5-methylphenyl | F |
| 095 | CH₃ | 2-fluoro-5-methylphenyl | CN |
| 096 | CH₃ | 2-fluoro-5-methylphenyl | OCH₂CF₃ |
| 097 | CH₃ | 2-fluoro-6-methylphenyl | F |
| 098 | CH₃ | 2-fluoro-6-methylphenyl | CN |
| 099 | CH₃ | 2-fluoro-6-methylphenyl | OCH₂CF₃ |
| 100 | CH₃ | 2-chloro-3-methylphenyl | F |
| 101 | CH₃ | 2-chloro-3-methylphenyl | CN |
| 102 | CH₃ | 2-chloro-3-methylphenyl | OCH₂CF₃ |
| 103 | CH₃ | 2-chloro-4-methylphenyl | F |
| 104 | CH₃ | 2-chloro-4-methylphenyl | CN |
| 105 | CH₃ | 2-chloro-4-methylphenyl | OCH₂CF₃ |
| 106 | CH₃ | 2-chloro-5-methylphenyl | F |
| 107 | CH₃ | 2-chloro-5-methylphenyl | CN |
| 108 | CH₃ | 2-chloro-5-methylphenyl | OCH₂CF₃ |
| 109 | CH₃ | 2-chloro-6-methylphenyl | F |
| 110 | CH₃ | 2-chloro-6-methylphenyl | CN |
| 111 | CH₃ | 2-chloro-6-methylphenyl | OCH₂CF₃ |
| 112 | CH₃ | 4-fluoro-2-methylphenyl | F |
| 113 | CH₃ | 4-fluoro-2-methylphenyl | CN |
| 114 | CH₃ | 4-fluoro-2-methylphenyl | OCH₂CF₃ |
| 115 | CH₃ | 4-chloro-2-methylphenyl | F |
| 116 | CH₃ | 4-chloro-2-methylphenyl | CN |
| 117 | CH₃ | 4-chloro-2-methylphenyl | OCH₂CF₃ |
| 118 | CH₃ | 2,4,6-trifluorophenyl | F |
| 119 | CH₃ | 2,4,6-trifluorophenyl | CN |
| 120 | CH₃ | 2,4,6-trifluorophenyl | OCH₂CF₃ |
| 121 | CH₃ | 2,3,6-trifluorophenyl | F |
| 122 | CH₃ | 2,3,6-trifluorophenyl | CN |
| 123 | CH₃ | 2,3,6-trifluorophenyl | OCH₂CF₃ |
| 124 | CH₃ | 2,3,4-trifluorophenyl | F |
| 125 | CH₃ | 2,3,4-trifluorophenyl | CN |
| 126 | CH₃ | 2,3,4-trifluorophenyl | OCH₂CF₃ |
| 127 | CH₃ | 2,4,6-trichlorophenyl | F |
| 128 | CH₃ | 2,4,6-trichlorophenyl | CN |
| 129 | CH₃ | 2,4,6-trichlorophenyl | OCH₂CF₃ |
| 130 | CH₃ | 2,3,6-trichlorophenyl | F |
| 131 | CH₃ | 2,3,6-trichlorophenyl | CN |
| 132 | CH₃ | 2,3,6-trichlorophenyl | OCH₂CF₃ |
| 133 | CH₃ | 2,3,4-trichlorophenyl | F |
| 134 | CH₃ | 2,3,4-trichlorophenyl | CN |
| 135 | CH₃ | 2,3,4-trichlorophenyl | OCH₂CF₃ |
| 136 | CH₃ | 2,6-difluoro-4-methoxyphenyl | F |
| 137 | CH₃ | 2,6-difluoro-4-methoxyphenyl | CN |
| 138 | CH₃ | 2,6-difluoro-4-methoxyphenyl | OCH₂CF₃ |
| 139 | CH₃ | 2,6-difluoro-4-trifluoromethoxyphenyl | F |
| 140 | CH₃ | 2,6-difluoro-4-trifluoromethoxyphenyl | CN |
| 141 | CH₃ | 2,6-difluoro-4-trifluoromethoxyphenyl | OCH₂CF₃ |
| 142 | CH₃ | 2,6-difluoro-4-trifluoromethylphenyl | F |
| 143 | CH₃ | 2,6-difluoro-4-trifluoromethylphenyl | CN |
| 144 | CH₃ | 2,6-difluoro-4-trifluoromethylphenyl | OCH₂CF₃ |
| 145 | CH₃ | 2,6-difluoro-4-cyanoyphenyl | F |
| 146 | CH₃ | 2,6-difluoro-4-cyanophenyl | CN |
| 147 | CH₃ | 2,6-difluoro-4-cyanophenyl | OCH₂CF₃ |
| 148 | CH₃ | 2,6-difluoro-4-methylphenyl | F |
| 149 | CH₃ | 2,6-difluoro-4-methylphenyl | CN |
| 150 | CH₃ | 2,6-difluoro-4-methylphenyl | OCH₂CF₃ |
| 151 | CH₃ | 2,6-dichloro-4-methoxyphenyl | F |
| 152 | CH₃ | 2,6-dichloro-4-methoxyphenyl | CN |
| 153 | CH₃ | 2,6-dichloro-4-methoxyphenyl | OCH₂CF₃ |
| 154 | CH₃ | 2,6-dichloro-4-trifluoromethoxyphenyl | F |
| 155 | CH₃ | 2,6-dichloro-4-trifluoromethoxyphenyl | CN |
| 156 | CH₃ | 2,6-dichloro-4-trifluoromethoxyphenyl | OCH₂CF₃ |
| 157 | CH₃ | 2,6-dichloro-4-trifluoromethylphenyl | F |
| 158 | CH₃ | 2,6-dichloro-4-trifluoromethylphenyl | CN |
| 159 | CH₃ | 2,6-dichloro-4-trifluoromethylphenyl | OCH₂CF₃ |
| 160 | CH₃ | 2,6-dichloro-4-cyanophenyl | F |
| 161 | CH₃ | 2,6-dichloro-4-cyanophenyl | CN |
| 162 | CH₃ | 2,6-dichloro-4-cyanophenyl | OCH₂CF₃ |
| 163 | CH₃ | 2,6-dichloro-4-methylphenyl | F |
| 164 | CH₃ | 2,6-dichloro-4-methylphenyl | CN |
| 165 | CH₃ | 2,6-dichloro-4-methylphenyl | OCH₂CF₃ |
| 166 | CH₃ | pentafluorophenyl | F |
| 167 | CH₃ | pentafluorophenyl | CN |
| 168 | CH₃ | pentafluorophenyl | OCH₂CF₃ |

where
a) 168 compounds of formula (I.a):

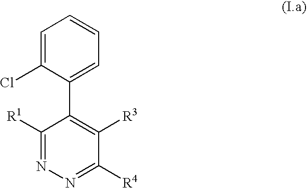

wherein R¹, R³ and R⁴ are as defined in Table 1.
b) 168 compounds of formula (I.b):

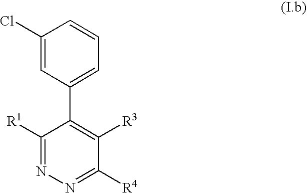

wherein R¹, R³ and R⁴ are as defined in Table 1.
c) 168 compounds of formula (I.c):

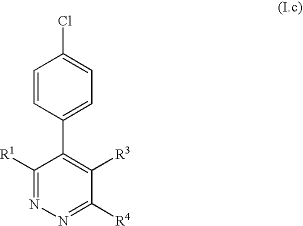

wherein R¹, R³ and R⁴ are as defined in Table 1.
d) 168 compounds of formula (I.d):

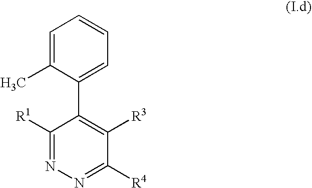

wherein R¹, R³ and R⁴ are as defined in Table 1.
e) 168 compounds of formula (I.e):

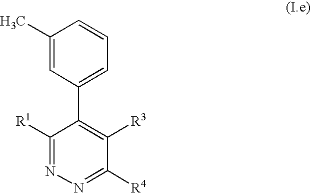

wherein R¹, R³ and R⁴ are as defined in Table 1.
f) 168 compounds of formula (I.f):

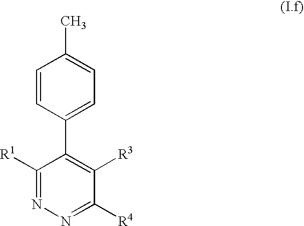

wherein R¹, R³ and R⁴ are as defined in Table 1.
g) 168 compounds of formula (I.g):

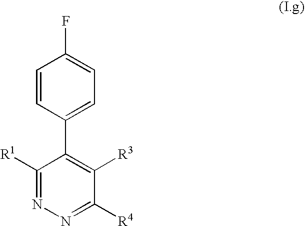

wherein R¹, R³ and R⁴ are as defined in Table 1.
h) 168 compounds of formula (I.h):

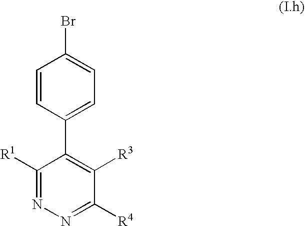

wherein R¹, R³ and R⁴ are as defined in Table 1.
i) 168 compounds of formula (I.i):

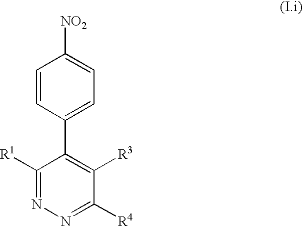

wherein R¹, R³ and R⁴ are as defined in Table 1.
j) 168 compounds of formula (I.j):

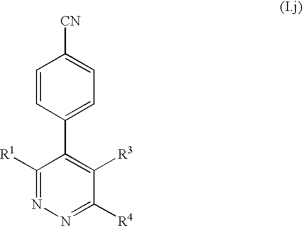

wherein R¹, R³ and R⁴ are as defined in Table 1.
k) 168 compounds of formula (I.k):

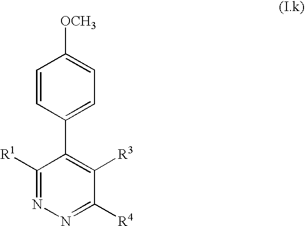

wherein R¹, R³ and R⁴ are as defined in Table 1.
1) 168 compounds of formula (I.l):

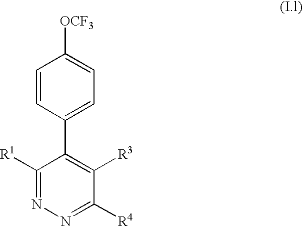

wherein R¹, R³ and R⁴ are as defined in Table 1.
m) 168 compounds of formula (I.m):

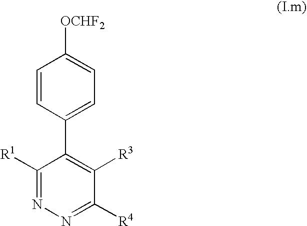

wherein R¹, R³ and R⁴ are as defined in Table 1.
n) 168 compounds of formula (I.n):

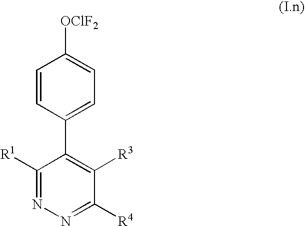

wherein R¹, R³ and R⁴ are as defined in Table 1.
o) 168 compounds of formula (I.o):

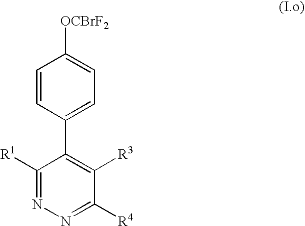

wherein R¹, R³ and R⁴ are as defined in Table 1.
p) 168 compounds of formula (I.p):

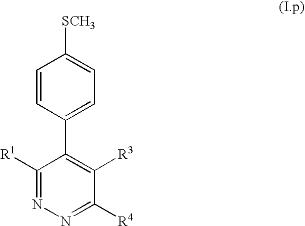

wherein R¹, R³ and R⁴ are as defined in Table 1.
q) 168 compounds of formula (I.q):

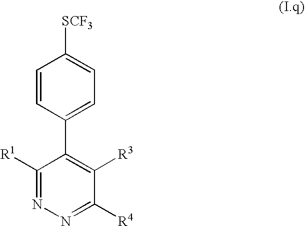

wherein R¹, R³ and R⁴ are as defined in Table 1.
r) 168 compounds of formula (I.r):

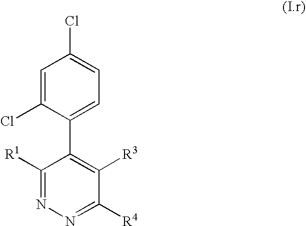

wherein R¹, R³ and R⁴ are as defined in Table 1.
s) 168 compounds of formula (I.s):

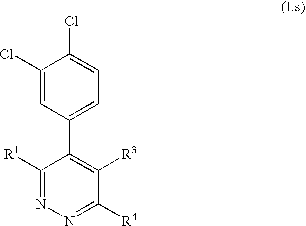

wherein R¹, R³ and R⁴ are as defined in Table 1.
t) 168 compounds of formula (I.t):

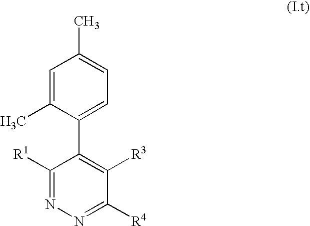

wherein R¹, R³ and R⁴ are as defined in Table 1.
u) 168 compounds of formula (I.u):

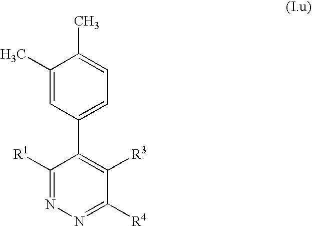

wherein R¹, R³ and R⁴ are as defined in Table 1.
v) 168 compounds of formula (I.v):

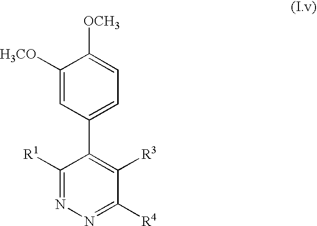

wherein R¹, R³ and R⁴ are as defined in Table 1.
w) 168 compounds of formula (I.w):

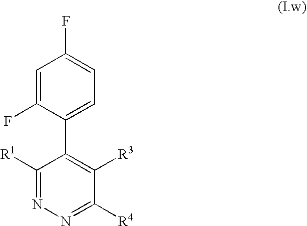

wherein R¹, R³ and Ware as defined in Table 1.
x) 168 compounds of formula (I.x):

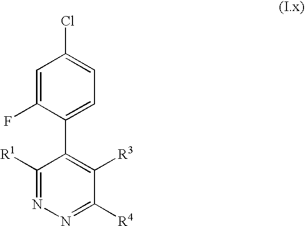

wherein R¹, R³ and R⁴ are as defined in Table 1.
y) 168 compounds of formula (I.y):

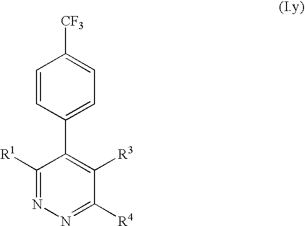

wherein R¹, R³ and R⁴ are as defined in Table 1.
z) 168 compounds of formula (I.z):

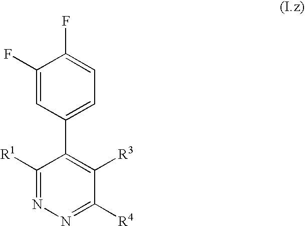

wherein R¹, R³ and R⁴ are as defined in Table 1.

Throughout this description, temperatures are given in degrees Celsius, m.p. means melting point, and “%” is percent by weight, unless corresponding concentrations are indicated in other units.

Table 3 shows selected melting point for compounds of Tables 1 and 2.

TABLE 3
Melting point for compounds of Tables 1 and 2

| Compound Number | m.p. (° C.) |
|---|---|
| I.c. 127 | 114-115 |
| I.c. 129 | 105-108 |

The compounds according to the present invention can be prepared according to the above-mentioned reaction schemes, in which, unless otherwise stated, the definition of each variable is as defined above for a compound of formula (I).

### Biological Examples

### Alternaria Solani/Tomato/Preventive (Action Against Alternaria on Tomato)

4 weeks old tomato plants cv. Roter Gnom are treated with the formulated test compound in a spray chamber. Two days after application tomato plants are inoculated by spraying a spore suspension on the test plants. After an incubation period of 4 days at 22° C./18° C. and 95% r. h. in a greenhouse the disease incidence is assessed.

Compounds of formula I according to the invention, in particular compounds I.c.127 and I.c.129 at 200 ppm inhibit fungal infestation in this test to at least 80%, while under the same conditions untreated control plants are infected by the phytopathogenic fungi to over 80%.

### Botrytis Cinerea/Tomato/Preventive (Action Against Botrytis on Tomato)

4 weeks old tomato plants cv. Roter Gnom are treated with the formulated test compound in a spray chamber. Two days after application tomato plants are inoculated by spraying a spore suspension on the test plants. After an incubation period of 3 days at 20° C. and 95% r. h. in a greenhouse the disease incidence is assessed.

Compounds of formula I according to the invention, in particular compound I.c.127 at 200 ppm inhibit fungal infestation in this test to at least 80%, while under the same conditions untreated control plants are infected by the phytopathogenic fungi to over 80%.

### Puccinia Recondita/Wheat/Preventive (Action Against Brown Rust on Wheat)

1 week old wheat plants cv. Arina are treated with the formulated test compound in a spray chamber. One day after application wheat plants are inoculated by spraying a spore suspension (1×105 uredospores/ml) on the test plants. After an incubation period of 1 day at 20° C. and 95% r. h. plants are kept for 10 days 20° C./18° C. (day/night) and 60% r.h. in a greenhouse. The disease incidence is assessed 11 days after inoculation.

Compounds of formula I according to the invention, in particular compound I.c.127 at 200 ppm inhibits fungal infestation in this test to at least 80%, while under the same conditions untreated control plants are infected by the phytopathogenic fungi to over 80%.

Magnaporthe Grisea (Pyricularia oryzae)/Rice/Preventive (Action Against Rice Blast)

3 weeks old rice plants cv. Koshihikari are treated with the formulated test compound in a spray chamber. Two days after application rice plants are inoculated by spraying a spore suspension (1×10^{5 conidia}/ml) on the test plants. After an incubation period of 6 days at 25° C. and 95% r. h. the disease incidence is assessed.

Compounds of formula I according to the invention, in particular compound I.c.127 at 200 ppm inhibit fungal infestation in this test to a least 80%, while under the same conditions untreated control plants are infected by the phytopathogenic fungi to over 80%.

### Prenophora Teres (Helminthosporium Teres)/Barley/Preventive (Action Against Net Blotch on Barley)

1-week-old barley plants cv. Regina are treated with the formulated test compound in a spray chamber. Two days after application barley plants are inoculated by spraying a spore suspension (2.6×10^{4 conidia}/ml) on the test plants. After an incubation period of 4 days at 20° C. and 95% r. h. the disease incidence is assessed.

Compounds of formula I according to the invention, in particular compound I.c.127 at 200 ppm inhibit fungal infestation in this test to at least 80%, while under the same conditions untreated control plants are infected by the phytopathogenic fungi to over 80%.

### Septoria Tritici/Wheat/Preventive (Action Against Septoria Leaf Spot on Wheat)

2 weeks old wheat plants cv. Riband are treated with the formulated test compound in a spray chamber. One day after application wheat plants are inoculated by spraying a spore suspension (10^{6 conidia}/ml) on the test plants. After an incubation period of 1 day at 22° C./21° C. and 95% r. h. plants are kept at 22° C./21° C. and 70% r.h. in a greenhouse. The disease incidence is assessed 16-18 days after inoculation.

Compounds of formula I according to the invention, in particular compounds I.c.127 and I.c.129 at 200 ppm inhibits fungal infestation in this test to at least 80%, while under the same conditions untreated control plants are infected by the phytopathogenic fungi to over 80%.

### Uncinula Necator/Drape/Preventive (Action Against Powdery Mildew on Grape)

5 weeks old grape seedlings cv. Gutedel are treated with the formulated test compound in a spray chamber. One day after application grape plants are inoculated by shaking plants infected with grape powdery mildew above the test plants. After an incubation period of 7 days at 24° C./22° C. and 70% r. h. under a light regime of 14/10 h (light/dark) the disease incidence is assessed.

Compounds of formula I according to the invention, in particular compounds I.c.127 and I.c.129 at 200 ppm inhibit fungal infestation in this test to at least 80%, while under the same conditions untreated control plants are infected by the phytopathogenic fungi to over 80%.

1. A compound of formula I: 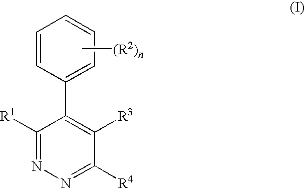
wherein
R¹ is hydrogen, C₁-C₆alkyl, C₁-C₆haloalkyl or C₃-C₆cycloalkyl;
R² is halogen, nitro, cyano, C₁-C₄alkyl, C₁-C₄haloalkyl, C₁-C₄alkoxy, C₁-C₄haloalkoxy, C₁-C₄alkylthio or C₁-C₄haloalkylthio;
R³ is an optionally substituted aryl;
R⁴ is fluoro, cyano, C₁-C₆haloalkyl, C₃-C₆cycloalkyl, C₁-C₆haloalkoxy, C₁-C₆alkylthio or C₁-C₆haloalkylthio; and
n is a whole number from 1 to 4;
or an agrochemically usable salt form thereof.

2. The compound according to claim 1 wherein R¹ is C₁-C₆alkyl, C₁-C₆haloalkyl or C₃-C₆cycloalkyl.

3. The compound according to either claim 1 wherein R² is halogen, nitro, cyano, C₁-C₃alkyl, C₁-C₃haloalkyl, C₁-C₃alkoxy, C₁-C₃haloalkoxy, C₁-C₃alkylthio or C₁C₃haloalkylthio.

4. The compound according to claim 1 wherein R³ is an optionally substituted phenyl.

5. The compound according to claim 1 wherein R⁴ is fluoro, cyano, C₁-C₆haloalkyl, C₁-C₆haloalkoxy, C₁-C₆alkylthio or C₁-C₆haloalkylthio.

6. The compound according to claim 1 wherein n is a whole number from 1 to 3.

7. The compound according to claim 1 wherein
R¹ is C₁-C₆alkyl or C₁-C₆haloalkyl;
R² is halogen, nitro, cyano, C₁-C₂alkyl, C₁-C₂haloalkyl, C₁-C₂alkoxy, C₁-C₂haloalkoxy, C₁-C₂alkylthio or C₁-C₂haloalkylthio;
R³ is 2-fluorophenyl, 2-chlorophenyl, 2-trifluoromethylphenyl, 2-methylphenyl, 2,3-difluorophenyl, 2,4-difluorophenyl, 2,5-difluorophenyl, 2,6-difluorophenyl; 2,3-dichlorophenyl, 2,4-dichlorophenyl, 2,5-dichlorophenyl, 2,6-dichlorophenyl, 2-chloro-3-fluorophenyl; 2-chloro-4-fluorophenyl, 2-chloro-5-fluorophenyl, 2-chloro-6-fluorophenyl, 3-chloro-2-fluorophenyl, 4-chloro-2-fluorophenyl, 5-chloro-2-fluorophenyl, 2-fluoro-3-trifluoromethylphenyl, 2-fluoro-4-trifluoromethylphenyl, 2-fluoro-5-trifluoromethylphenyl, 2-fluoro-6-trifluoromethylphenyl, 2-chloro-3-trifluoromethylphenyl, 2-chloro-4-trifluoromethylphenyl, 2-chloro-5-trifluoromethylphenyl, 2-chloro-6-trifluoromethylphenyl, 4-fluoro-2-trifluoromethylphenyl, 4-chloro-2-trifluoromethylphenyl, 2-fluoro-3-methylphenyl, 2-fluoro-4-methylphenyl, 2-fluoro-5-methylphenyl, 2-fluoro-6-methylphenyl, 2-chloro-3-methylphenyl, 2-chloro-4-methylphenyl, 2-chloro-5-methylphenyl, 2-chloro-6-methylphenyl, 4-fluoro-2-methylphenyl, 4-chloro-2-methylphenyl, 2,4,6-trifluorophenyl, 2,3,6-trifluorophenyl, 2,3,4-trifluorophenyl, 2,4,6-trichlorophenyl, 2,3,6-trichlorophenyl, 2,3,4-trichlorophenyl, 2,6-difluoro-4-methoxyphenyl, 2,6-difluoro-4-trifluoromethoxyphenyl, 2,6-difluoro-4-trifluoromethylphenyl, 2,6-difluoro-4-cyanophenyl, 2,6-difluoro-4-methylphenyl, 2,6-dichloro-4-methoxyphenyl, 2,6-dichloro-4-trifluoromethoxyphenyl, 2,6-dichloro-4-trifluoromethylphenyl, 2,6-dichloro-4-cyanophenyl, 2,6-dichloro-4-methylphenyl or pentafluorophenyl;
R⁴ is fluoro, cyano or C₁-C₆haloalkoxy; and
n is a whole number from 1 to 2.

8. The compound according to claim 1 wherein
R¹ is C₁-C₃alkyl;
R² is chloro, bromo, fluoro, nitro, cyano, methyl, C₁-C₂haloalkyl, methoxy, trifluoromethoxy, difluoromethoxy, chloro-difluoromethoxy, bromo-difluoromethoxy, methylthio or trifluoromethylthio;
R³ is 2-trifluoromethylphenyl, 2,4-dichlorophenyl, 2-chloro-6-fluorophenyl, 2,4,6-trifluorophenyl or 2,6-difluoro-4-methoxyphenyl;
R⁴ is fluoro, cyano or 2,2,2-trifluoroethoxy; and
n is a whole number from 1 to 2.

9. The compound according to claim 1 wherein
R¹ is methyl;
R² chloro;
R³ is 2,4,6-trichlorophenyl;
R⁴ is fluoro or 2,2,2-trifluoroethoxy; and
n is 1.

10. A compound selected from
4-(4-chlorophenyl)-6-fluoro-3-methyl-5-(2,4,6-trifluorophenyl)-pyridazine,
3-fluoro-6-methyl-5-p-tolyl-4-(2,4,6-trifluorophenyl)-pyridazine,
5-(4-chlorophenyl)-6-methyl-4-(2,4,6-trifluorophenyl)-pyridazine-3-carbonitrile,
6-methyl-5-p-tolyl-4-(2,4,6-trifluorophenyl)-pyridazine-3-carbonitrile,
4-(4-chlorophenyl)-3-methyl-6-(2,2,2-trifluoroethoxy)-5-(2,4,6-trifluorophenyl)-pyridazine,
3-methyl-4-p-tolyl-6-(2,2,2-trifluoroethoxy)-5-(2,4,6-trifluorophenyl)-pyridazine,
4-(2-chloro-6-fluorophenyl)-5-(4-chlorophenyl)-3-fluoro-6-methyl-pyridazine,
4-(2-chloro-6-fluorophenyl)-3-fluoro-6-methyl-5-p-tolyl-pyridazine,
4-(2-chloro-6-fluorophenyl)-5-(4-chlorophenyl)-6-methyl-pyridazine-3-carbonitrile,
4-(2-chloro-6-fluorophenyl)-6-methyl-5-p-tolyl-pyridazine-3-carbonitrile,
4-(2-chloro-6-fluorophenyl)-5-(4-chlorophenyl)-6-methyl-3-(2,2,2-trifluoroethoxy)-pyridazine, and
4-(2-chloro-6-fluorophenyl)-6-methyl-5-p-tolyl-3-(2,2,2-trifluoroethoxy)-pyridazine.

11. A process for the preparation of a compound of formula I.2, 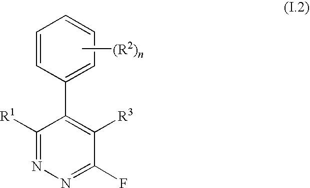
wherein R¹, R², R³ and n are as defined for compound of formula I, which comprises reacting a compound of formula I.1, 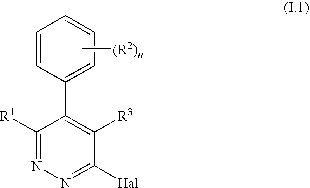
wherein R¹, R², R³ and n are as defined for compound of formula I and Hal is chlorine or bromine, with an inorganic fluoride.

12. A process for the preparation of a compound of formula I.3, 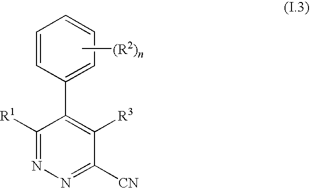
wherein R¹, R², R³ and n are as defined for compound of formula I, which comprises reacting a compound of formula I.1, 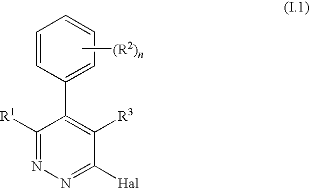
wherein R¹, R², R³ and n are as defined for compound of formula I and Hal is chlorine or bromine, with an inorganic cyanide.

13. A process for the preparation of a compound of formula I.4, 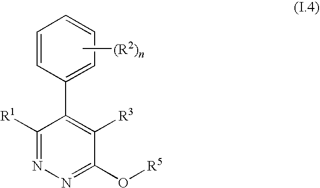
wherein R¹, R², R³ and n are as defined for compound of formula I and R⁵ is C₁-C₆haloalkyl, which comprises reacting a compound of formula I.1, 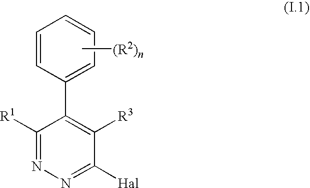
wherein R¹, R², R³ and n are as defined for compound of formula I and Hal is halogen with an alcohol R⁵OH, wherein R⁵ is C₁-C₆haloalkyl, and a base or with a sodium alkoxide NaOR⁵, wherein R⁵ is C₁-C₆haloalkyl.

14. A process for the preparation of a compound of formula I.5, 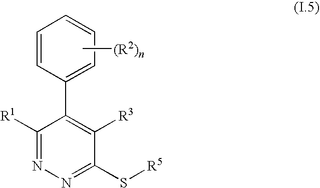
wherein R¹, R², R³ and n are as defined for compound of formula I and R⁵ is C₁-C₆alkyl or C₁-C₆haloalkyl, which comprises reacting a compound of formula I.1, 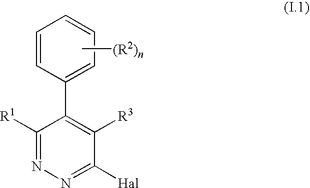
wherein R¹, R², R³ and n are as defined for compound of formula I and Hal is halogen with a thiol R⁵SH, wherein R⁵ is C₁-C₆alkyl or C₁-C₆haloalkyl, and a base or with a sodium thioalkoxide NaSR⁵, wherein R⁵ is C₁-C₆alkyl or C₁-C₆haloalkyl.

15. A fungicidal composition for controlling or protecting against phytopathogenic microorganisms, comprising as active ingredient at least one compound as defined in claim 1, in free form or in agrochemically usable salt form, and at least one adjuvant.

16. The composition according to claim 15, which comprises at least one additional fungicidally active compound, preferably selected from the group consisting of azoles, pyrimidinyl carbinoles, 2-amino-pyrimidines, morpholines, anilinopyrimidines, pyrroles, phenylamides, benzimidazoles, dicarboximides, carboxamides, strobilurines, dithiocarbamates, N-halomethylthiotetrahydrophthalimides, copper-compounds, nitrophenols, organo-phosphorus-derivatives, pyridazines, triazolopyrimidines or benzamides.

17. (canceled)

18. A method of controlling or preventing an infestation of crop plants, harvested food crops or non-living materials by phytopathogenic or spoilage microorganisms or organisms potentially harmful to man, which comprises the application of a compound as defined in claim 1, as active ingredient to the plant, to parts of the plants or to the locus thereof, to seeds or to any part of the non-living materials.

19. The method according to claim 18, wherein the phytopathogenic microorganisms are fungal organisms.
